(19) United States
(12) Patent Application Publication
(10) Pub. No.: US 2024/0238487 A1
(43) Pub. Date: Jul. 18, 2024

(54) System for Detecting Breast Pump Properties

(71) Applicant: MEDELA HOLDING AG, Baar (CH)

(72) Inventor: Michael Larsson, BAAR (CH)

(21) Appl. No.: 18/564,962

(22) PCT Filed: Jun. 8, 2022
(86) PCT No.: PCT/EP2022/065513

§ 371 Date: Nov. 28, 2023

(30) Foreign Application Priority Data
Jun. 9, 2021 (EP) 21178588.6

(51) Int. Cl. A61M 1/06

(52) U.S. Cl. CPC A61M 1/06; A61M 2205/3303; A61M 2205/3327; A61M 2205/3331; A61M 2205/3389; A61M 2205/3553; A61M 2205/3584; A61M 2205/3592; A61M 2205/502; A61M 2205/60; A61M 2205/8206; A61M 2209/088; A61M 2210/1007; A61M 2230/04

(57) ABSTRACT

The present invention relates to a system for detecting properties of at least one breast pump remotely. The system comprises at least one breast pump unit and a sensor for detecting at least one property of the at least one breast pump unit.

The present invention relates to a system for detecting properties of at least one breast pump remotely.

Breast pumps for extracting or expressing breastmilk from a woman's breasts for later use by an infant typically include a funnel-shaped hood, or breast shield, that is placed over the nipple and a substantial portion of the breast. A reduced pressure or vacuum is intermittently generated in the hood in a manner which causes milk to be expressed from the breast within the hood. The milk then typically flows from the hood to a storage container for later use.

Modern breast pumps are motor-driven and usually contain an electronic control unit for controlling the operation of the motor that generates the reduced pressure or vacuum.

The properties of the breast pump have to be monitored continuously during its operation. For example, the fill level of the storage container must be monitored to prevent an overflow of the container. Similarly, operation of the motor must be monitored so that malfunctions can be detected quickly. In the case of battery-powered motors, the battery charge also has to be monitored.

Breast pumps may also be used as part of a system combining a plurality of individual breast pump units. If a malfunction is occurs in such a system, it is important to determine which of the plurality of breast pump units is affected.

In conventional breast pumps, monitoring the breast pump's properties requires direct access to the breast pump. This is inconvenient for the user as well as for medical staff in a clinical setting.

Therefore, the present invention aims at providing a system for detecting properties of at least one breast pump remotely. Specifically, the system should allow detecting properties of at least one breast pump in a system comprising a plurality of breast pumps. For example, the system should allow detecting the position of an individual breast pump within a plurality of breast pumps.

To solve this problem, the invention relates to a system comprising at least one breast pump unit and a sensor for detecting at least one property of the at least one breast pump unit.

According to the invention, the sensor is configured to detect at least one property of the at least one breast pump unit and to transmit a signal indicative of said property. By using the sensor according to the invention, it is possible to monitor the properties of the at least one breast pump unit remotely. For example, the system allows monitoring the properties of the at least one breast pump during use without the need for having to look at the breast pump directly. Thereby, the breast pump can be covered by clothing (e.g. it can be worn beneath a shirt) and its properties can be monitored without having to remove the clothing. Another advantage of the invention is that the system allows monitoring the properties of a plurality of breast pumps located in a storage facility, for example, in a hospital or a warehouse, without having to inspect each breast pump unit directly.

The at least one breast pump unit preferably comprises a breast shield for applying a negative pressure to the breast of a user and extracting milk from the breast, and a container for receiving the expressed milk. The at least one breast pump unit may further comprises a motor for generating the negative pressure applied to the breast and a power supply for providing electric energy to the motor. Preferably, the power supply is a rechargeable battery. More preferably, all of these components are integrated into a single housing to form a breast pump unit that can be directly attached to a user's breast.

Preferably, the at least one breast pump unit may be incorporated into a bra cup (in-bra breast pump). In this case, the system offers the advantage of monitoring the properties of the breast pump unit without having to open the bra or having to disassemble the breast pump unit from the bra cup.

The sensor may include a detection unit that is positioned on the breast pump unit and is configured to detect any property of the at least one breast pump unit. For example, the detection unit is configured to detect the fill level of a container attached to the breast pump unit, the status of the power supply of the breast pump unit, the operation parameters of the breast pump motor, and/or the level of negative pressure applied to the breast. As another example, the property may also be an identification code for identifying the particular breast pump unit.

The sensor may further be configured to convert the detected property into a signal indicative of the property. The signal may be a qualitative signal indicating, for example, whether or not the fill level of the container has reached its maximum capacity, or it may be a quantitative signal indicating, for example, the amount of electric charge left in the power supply or the amount of liquid collected in the container.

The signal may be transmitted by a physical connection, such as an electric or optical cable connection, or it may be transmitted wirelessly. Preferably, the signal is transmitted wirelessly, for example, by using an optical or acoustic signal or by transmitting an electronic signal through a wireless connection, such as a wireless local area network (WLAN) connection, a Bluetooth connection or a radio-frequency identification (RFID) system.

In one embodiment, the sensor comprises at least one sender positioned on the at least one breast pump unit and at least one receiver positioned remotely from the breast pump unit, wherein the sender transmits a signal indicative of at least one property of the breast pump unit to the receiver. Preferably, the sender transmits the signal wirelessly to the receiver.

In a preferred embodiment, the sender is a RFID tag and the receiver is a RFID reader. The RFID tag transmits a signal indicative of the identity of the breast pump unit to the RFID reader. Thereby, a breast pump unit can be identified among a plurality of breast pump units. Further, the position of the breast pump unit relative to the RFID reader can be determined, for example by evaluating the strength and/or direction of the received signal.

In another embodiment, the sensor comprises at least one acoustic sender and at least one acoustic receiver, wherein the acoustic sender generates and transmits an acoustic signal indicative of at least one property of the breast pump to the acoustic receiver. This may, for example, be used to determine the position of the acoustic sender relative to the acoustic receiver.

In one embodiment, the system comprises a plurality of (two or more) breast pump units. For example, the system may comprise two breast pump units, preferably in-bra breast pumps, that can be simultaneously attached to the two breasts of a user. As another example, the system may comprise a plurality of breast pump units located in the storage of a hospital or a warehouse.

In one embodiment, the system is configured to determine the relative position of each of a plurality of breast pump units. For example, the system may be configured to determine whether a given breast pump unit is located left or right from another breast pump unit. Preferably, the signal is indicative of the position of the breast pump unit and one or more further properties of the breast pump unit. For example, the signal may be indicative of the position of each breast pump unit and the fill level of each of the containers attached to each individual breast pump unit.

In case of a plurality of breast pump units, each breast pump unit may be equipped with a sender for transmitting a signal indicative of at least one property of the respective breast pump unit and a receiver for receiving the signal from one or more other breast pump units. Thereby, it is possible to exchange information between different breast pump units. For example, it is possible for a given breast pump unit to determine its position relative to another breast pump unit.

In a preferred embodiment, each of a plurality of breast pump units comprises a RFID tag, a RFID reader and a RFID shield. The RFID shield of each breast pump unit may be configured so that the signal of the RFID tag is transmitted into one predetermined direction only. Alternatively, the RFID shield of each breast pump unit is configured to block the reception of signal on one side of the RFID reader. For example, the RFID shield may be placed next to the RFID reader so that the RFID reader only receives signal from one side of the breast pump unit. Thereby, the RFID reader of one breast pump unit can determine the relative position of the RFID tag of a neighboring breast pump unit. Thus, the system may determine whether a given breast pump unit is located left or right from another breast pump unit.

In another embodiment, each of a plurality of breast pump units comprises an acoustic sender and at least one acoustic receiver, preferably at least two acoustic receivers. Thereby, the acoustic receiver on one breast pump unit may detect the signal emitted by the acoustic sender of another breast pump unit. This allows to determine the position of one breast pump unit relative to another. Preferably, each breast pump unit comprises at least two acoustic detectors and the signal transmitted from the acoustic sender of another breast pump unit is analyzed using timeof-flight difference or sound difference to determine the relative position of the other breast pump unit.

In another embodiment, each of a plurality of breast pump units comprises an acoustic receiver configured to detect the user's heartbeat. This allows the system to measure a difference in the heartbeat signal detected by each acoustic receiver. Thereby, the system can determine, for example, a time-of-flight difference or a sound level difference between the signals received by two acoustic receivers. This allows the system to determine which breast pump unit is located closer to a user's heart and is therefore located on the left body side of a user. Thus, the system can determined the position of each breast pump unit relative to each other.

In another embodiment, each breast pump unit is configured to be positioned at a defined tilting angle on a user's breast, where this tilting angle depends on whether the breast pump unit is positioned on the right or the left breast of a user, and each breast pump unit comprises a tilt sensor for measuring the tilting angle. For example, each breast pump unit may be configured such that it is inclined towards the center line between a user's breasts, if it is positioned on a user's breast. Thus, the tilt sensor will measure a different tilting angle depending on whether the breast pump unit is positioned on the left or the right breast. By analyzing the tilting angle provided by the tilting sensor, the system is able to determine the relative position of each breast pump unit.

In one embodiment, the system comprises a control unit for receiving and processing the signal from the sensor. In case the system comprises a plurality of breast pump units, the control unit may be configured to receive and process the signals from all the breast pump units. For example, the control unit is an electronic data processing device, such as a server, a desktop PC, a laptop PC, a tablet, a smartphone, or a smart watch. Preferably, the control unit has a display for displaying information on the property detected by the sensor.

The transmission of the sensor signal may be controlled automatically by the system or manually by the user. For example, the breast pump unit may comprise a switch, with which the user may switch the sensor on or off. Alternatively, the sensor may be switched on or off remotely through the above-mentioned control unit.

The invention is further illustrated by the following figures:

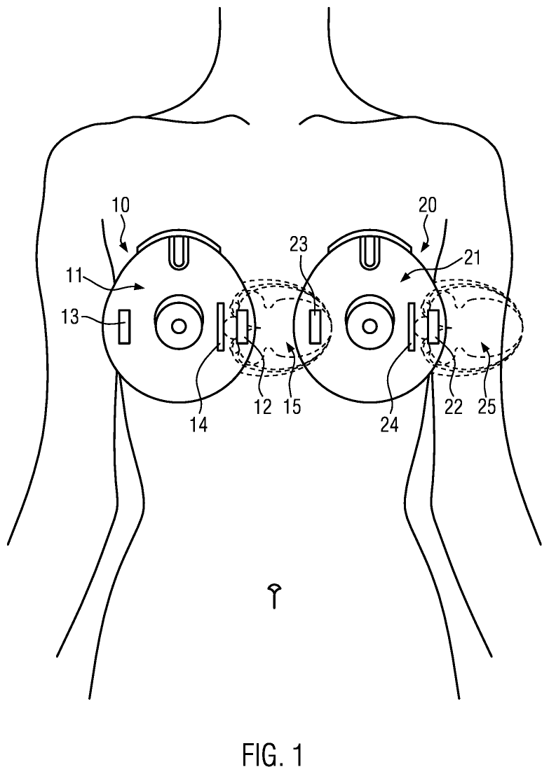

FIG. 1: A schematic drawing of a system comprising two breast pump units according to one embodiment of the invention.

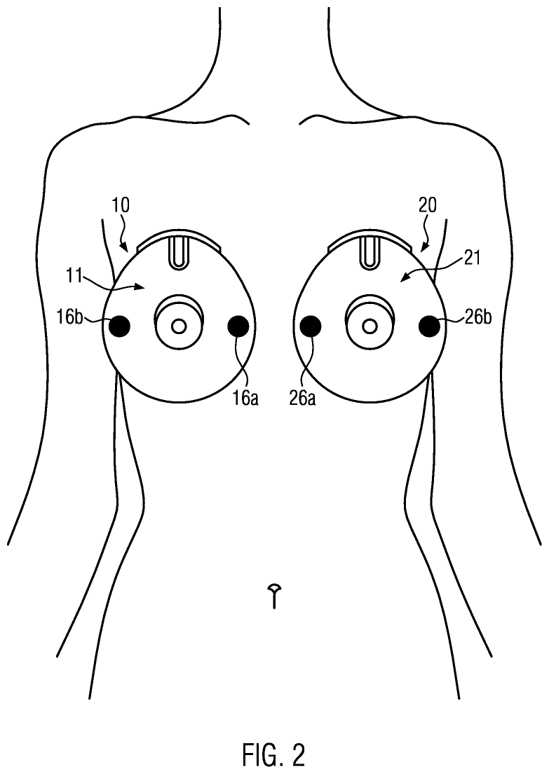

FIG. 2: A schematic drawing of a system comprising two breast pump units according to another embodiment of the invention.

FIG. 1 is a schematic drawing of a system comprising two breast pump units 10, 20 according to one embodiment of the present invention. In this embodiment, the system is configured to determine the position of each breast pump unit relative to another breast pump unit by using electromagnetic radio frequency signals.

Each breast pump unit 10, 20 is formed by a breast shield 11, 21. Each breast shield 11, 21 comprises a RFID reader 12, 22 a RFID tag 13, 23 and a RFID shield 14, 24. The RFID readers 12, 22 each emit an electromagnetic field 15, 25. Because the RFID readers 12, 22 are positioned close to the respective RFID shields 14, 24 of the same breast shield 11, 21, the electromagnetic fields 15, 25 are only emitted in one direction. In the example shown in FIG. 1, the electromagnetic fields 15, 25 are only emitted towards the left side from the user's perspective. In the other direction, the RFID shields 14, 24 block the transmission of the electromagnetic fields 15, 25.

In the embodiment shown in FIG. 1, the RFID tag 23 on the breast shield 21 receives the electromagnetic field 15 emitted from the RFID reader 12 on the breast shield 11. Thus, the identification signal from the RFID tag 23 can be transmitted to the RFID reader 12. RFID reader 12 thus detects that RFID tag 23 is positioned in close proximity. On the other hand, RFID reader 22 does not detect a signal from any RFID tag. Similarly, no RFID reader detects the presence of the RFID tag 13 on the first breast shield 11. Thereby, the system can determine that the breast pump unit 10 is positioned on the right breast whereas breast pump unit 20 is positioned on the left breast.

FIG. 2 is a schematic drawing of a system comprising two breast pump units 10, 20 according to another embodiment of the present invention. In this embodiment, the system is configured to determine the position of each breast pump unit relative to another breast pump unit by using acoustic signals.

Each breast pump unit 10, 20 is formed by a breast shield 11, 21. Each breast shield 11, 21 comprises a pair of microphones 16, 26 and an acoustic signal generator (not shown). Each pair of microphones comprises a proximal microphone 16a, 26a and a distal microphone 16b, 26b. The microphones 16, 26 are positioned symmetrical on opposing sides of the breast shields 11, 21. In the example shown in FIG. 1, the microphones 16, 26 are positioned left and right from the center of the respective breast shields 11, 26. Note that the microphones are aligned horizontally, because the breast shields 11, 21 are positioned at a tilting angle of 0°. It is, however, also possible to position the breast shields 11, 21 at an inclined position, i.e. at a tilting angle of greater than or less than 0°. The microphones 16, 26 are configured to detect the acoustic signal emitted by the acoustic signal generator of the other breast pump unit 10, 20. The microphones 16, 26 are preferably placed behind the outer walls of the breast shields 11, 21, i.e. between the respective outer wall and the user's skin. This facilitates cleaning the breast shields and is an important feature from a hygienic standpoint.

The system according to FIG. 2 is configured to measure the time-of-flight difference and/or the sound level difference between each microphone of a given pair. For example, if the microphones 16 on the breast shield 11 receive the acoustic signal from the acoustic signal generator on the breast shield 21, the proximal microphone 16a, i.e. the microphone that is closer to the breast shield 21, will receive the acoustic signal earlier than the distal microphone 16b. Thus, the time-of-flight is shorter for the proximal microphone 16a than for the distal microphone 16b. Similarly, the sound level of the acoustical signal, i.e. the signal intensity, detected by the proximal microphone 16a will be higher than that detected by the distal microphone 16b due to the larger distance between the acoustic signal generator and the distal microphone 16b. Based on the differences in the time-of-flight and/or the sound level, the system can determine that the respective proximal microphones 16a, 26a are closer the neighboring breast shields 11, 21 than the respective distal microphones 16b, 26b. Thereby, the system can determine the relative position of the two breast pump units 11, 20.

The system of FIG. 2 can further be modified by configuring the microphones 16, 26 so that they detect the heartbeat of the user. In this embodiment, no separate acoustic signal generator is required and it is sufficient if each breast shield 11, 22 only comprises a single microphone 16, 26 instead of a pair of microphones. The single microphones must be configured to detect the heartbeat of the user, i.e. they must preferably be positioned close to the user's skin. The system is configured to measure time-of-flight difference and/or the sound level difference between the two microphones 16, 26. The microphone 26 that is located closer to the left body side of the user will detect the heartbeat signal earlier and with a higher sound level than the microphone 16 that is located closer to the right body side. Thereby, the system can determine which breast pump unit 10, 20 is positioned on the left side and which is positioned on the right side of the user.

### LIST OF REFERENCE SIGNS

10 breast pump unit
11 breast shield
12 RFID reader
13 RFID tag
14 RFID shield
15 electromagnetic field
16 microphones
16a proximal microphone
16b distal microphone
20 breast pump unit
21 breast shield
22 RFID reader
23 RFID tag
24 RFID shield
25 electromagnetic field
26 microphones
26a proximal microphone
26b distal microphone

What is claimed is:

1. A system comprising at least one breast pump unit and a sensor for detecting at least one property of the at least one breast pump unit.

2. The system according to claim 1, wherein the sensor is configured to transit a signal indicative of the at least one property of the at least one breast pump unit.

3. The system according to claim 1, wherein the sensor includes a detection unit that is positioned on the breast pump unit and is configured to detect at least one property of the at least one breast pump unit.

4. The system according to claim 3, wherein the detection unit is configured to detect the fill level of a container attached to the breast pump unit, the status of a power supply of the breast pump unit, the operation parameters of a breast pump motor, and/or the level of negative pressure applied by the breast pump unit to the breast.

5. The system according to claim 1, wherein the system comprises a plurality of breast pump units and is configured to detect the position of each of the plurality of breast pump units.

6. The system according to claim 1, wherein the sensor comprises at least one sender positioned on the at least one breast pump unit and at least one receiver positioned remotely from the breast pump unit, wherein the sender transmits a signal indicative of at least one property of the breast pump unit to the receiver.

7. The system according to claim 6, wherein the at least one breast pump unit is equipped with a sender for transmitting a signal indicative of at least one property of the respective breast pump unit and a receiver for receiving the signal from one or more other breast pump units.

8. The system according to claim 6, wherein the sender is a RFID tag and the receiver is a RFID reader.

9. The system according to claim 8, wherein the breast pump unit further comprises a RFID shield configured so that the signal of the RFID tag is transmitted into one predetermined direction only.

10. The system according to claim 6, wherein the sender is an acoustic sender and the receiver is an acoustic receiver.

11. The system according to claim 10, wherein the breast pump unit comprises an acoustic sender and at least two acoustic receivers.

12. The system according to claim 1, wherein the system further comprises a control unit for receiving and processing the signal from the sensor.

13. The system according to claim 1, wherein transmission of the sensor signal may be controlled automatically by the system or manually by the user.

14. The system according to claim 1, wherein the breast pump unit comprises an acoustic sensor for measuring the heartbeat of a user.

15. The system according to claim 1, wherein the breast pump unit is configured to be positioned at a defined tilting angle on a user's breast, where this tilting angle depends on whether the breast pump unit is positioned on the right or the left breast of a user, and the breast pump unit comprises a tilt sensor for measuring the tilting angle.

16. The system according to claim 8, wherein the breast pump unit further comprises a RFID shield configured so that the signal of the RFID tag is transmitted so that the reception of signal is blocked on one side of the RFID reader.
